(19) United States
(12) Patent Application Publication
(10) Pub. No.: US 2017/0065728 A1
(43) Pub. Date: Mar. 9, 2017

(54) SELF-POWERED ENZYME MICROPUMPS

(71) Applicant: THE PENN STATE RESEARCH FOUNDATION, University Park, PA (US)

(72) Inventors: Ayusman Sen, State College, PA (US); Samudra Sengupta, Hillsboro, OR (US); Debabrata Patra, University Park, PA (US); Isamar Ortiz-Rivera, State College, PA (US)

(21) Appl. No.: 15/123,348

(22) PCT Filed: Mar. 3, 2015
(86) PCT No.: PCT/US15/18477

§ 371 Date: Sep. 2, 2016

Related U.S. Application Data
(60) Provisional application No. 61/947,272, filed on Mar. 3, 2014.
(60) Provisional application No. 62/047,913, filed on Sep. 9, 2014.

(51) Int. Cl. A61K 49/00; C12Q 1/54; A61K 9/00; C12N 11/14; A61K 9/06; A61K 38/28

(52) U.S. Cl. CPC A61K 49/0004; A61K 9/06; A61K 38/28; A61K 9/0097; A61K 9/0024; C12N 11/14; C12Y 301/03; C12Q 1/54; C12Y 101/03004; G01N 2610/00; G01N 2333/904

(57) ABSTRACT

Drug delivery devices, sensors, and micropumps provided herein can utilize a reaction of an analyte triggered by an enzyme to drive fluid flow. In some cases, a drug delivery device can include a reservoir including a drug (e.g., insulin) and have an enzyme (e.g., glucose oxidase) positioned adjacent to said reservoir. The enzyme can catalyze a reaction of said analyte to drive a fluid flow adjacent to said reservoir to increase a release of the drug from said reservoir. A sensor for an analyte can include an enzyme bound to a surface and a flow meter to detect a flow of fluids adjacent to said surface. A self-powered enzyme micropump provided herein can provide precise control over flow rate in response to specific signals.

### FEDERALLY SPONSORED RESEARCH OR DEVELOPMENT

This invention was made with the National Science Foundation under Grant No. DMR-0820404 and, in part, by the Defense Threat Reduction Agency (HDTRA1-13-1-0039), with Pennsylvania State University Materials Research Institute Nanofabrication. This publication is also based on work supported by Award No. RUP1-7078-PE-12 of the U.S. Civilian Research & Development Foundation (CRDF Global) and by the National Science Foundation under Cooperative Agreement No. OISE-9531011 (joint grant with Ural Branch of the Russian Academy of Sciences) and acknowledges a National Science Foundation Fellowship (DGE-1255832). The Government has certain rights in the invention.

### FIELD

This disclosure generally relates to drug delivery devices, sensors and self-powered micropumps.

### BACKGROUND

Detection of substances in situ is desirable to reduce the need for extraneous equipment or devices. Typically, in order to measure a substance in a body, a sample is drawn from the body and measured using an external device. Furthermore, if any remedial action is deemed appropriate, a second device is utilized to introduce a therapeutic drug into the body. For example, for patients with diabetes who take insulin, the process of treating their condition is quite complex. They must keep track of the amount of carbohydrates and other nutrients that they ingest; they must monitor capillary blood glucose values by repeated lancing of fingers or other body sites; and they must take into consideration the amount of exercise in which they engage. They must take into consideration all these factors in order to compute the doses of insulin that they administer regularly. If the glucose concentration is not well controlled and is chronically elevated, they run a risk of developing long term complications such as disease of the eyes, kidneys, nerves, feet and heart. If their blood glucose concentration falls too low, they run a risk of, for example, experiencing seizures, coma and nervous system damage.

For all these reasons, a system that could deliver the correct amounts of insulin with little or no patient interaction would be helpful to a person with insulin-treated Type 1 or Type 2 diabetes. However, automated pancreas systems have been quite cumbersome to date. For example, in the late 1970's a large device known as the BIOSTATOR was developed and was able to measure glucose on a continuous or near-continuous basis by withdrawing and measuring venous blood glucose values. See Fogt E J, Dodd L M, Jenning E M, Clemens A H, Development and evaluation of a glucose analyzer for a glucose controlled insulin infusion system (BIOSTATOR), Clin. Chem., 1978 August; 24(8):1366-72. In addition, the BIOSTATOR was able to administer insulin. Because of its size, the BIOSTATOR was relegated to a research tool and was never able to achieve widespread use among people with diabetes.

In more recent years, other attempts have been made to integrate a glucose sensor and an insulin infusion device. One such system was described by Hovorka and colleagues (Hovorka R, Chassin L J, Wilinska M E, et al., Closing the Loop, the Adicol Experience, Diabetes Technol. Ther., 2004 June; 6(3):307-18). In this system, a temporarily-implanted needle-type glucose sensor (microdialysis-type) was combined with a hand held computer and a belt-worn insulin pump in order to close the loop. One limitation of a microdialysis-type sensor is that it is a complicated device that requires fluid delivery into the microdialysis catheter, and fluid removal from the microdialysis catheter.

Steil and colleagues have also described a complex closed loop system, in which an intravenous sensor or subcutaneous sensor is combined with a fully-implantable or an external insulin pump and a computer (Steil G M, Panteleon A E, and Rebrin K, Closed-loop insulin delivery—the path to physiological glucose control, Adv Drug Deliv Rev, 2004 Feb. 10; 56(2): 125-44). However, such a system requires two separate units: one for the insulin pump (and catheter) and one for the sensing apparatus (which may use a separate catheter for sensing).

In other environments, such as sensing of toxic compounds, similar desirability for sensing of the analyte in situ and delivery of drugs may arise. For example, it is known that some organophosphate (OP) compounds have been used as chemical warfare agents for their ability to bind irreversibly to acetylcholinesterase, an enzyme involved in the nerve-signaling pathway Inhibition of this enzyme leads to over-stimulation of nerves and muscles, which can end in paralysis, convulsions and heart failure. Early detection of OP poisoning is key for saving the life of a victim, and if combined with the appropriate treatment, it could avoid secondary effects of the poisoning, such as brain damage.

To design such a device that could incorporate both sensing and transport, without the need of external equipment or power sources, complicated mechanisms of action and/or sizable mechanical parts, micropumps seem to be a suitable option. A micropump is any kind of small pump, including pumps with functional dimensions in the micrometer range. Such pumps are of special interest in microfluidic research, and have become available for industrial product integration in recent years. Their miniaturized overall size, potential cost and improved dosing accuracy compared to existing miniature pumps fuel the growing interest for this innovative kind of pump.

### SUMMARY

A self-powered enzyme micropump provided herein can provide precise control over flow rate in response to specific signals. In some cases, self-powered enzyme micropumps provided herein can be ATP-independent. In some cases, self-powered enzyme micropumps provided herein can be non-mechanical. In some cases, self-powered enzyme micropumps provided herein can be surface-immobilized. In some cases, self-powered enzyme micropumps provided herein can include an enzyme selected from catalase, lipase, urease, glucose oxidase, and combinations thereof. In some cases, self-powered enzyme micropumps provided herein can provide a flow driven by a fluid density-gradient generated by an enzymatic reaction. In some cases, self-powered enzyme micropumps provided herein can increase the flow velocity with increasing substrate concentration and reaction rate. In some cases, self-powered enzyme micropumps provided herein can be triggered by the presence of specific analytes and can act as both a sensor and a pump. In some cases, self-powered enzyme micropumps provided herein can autonomously deliver small molecules and proteins in response to specific chemical stimuli. For example, self-powered enzyme micropumps provided herein can, in some cases, be used to release insulin in response to the presence of glucose.

In some cases, self-powered enzyme micropumps provided herein can include simple pattern of enzymes on a surface. In some cases, self-powered enzyme micropumps provided herein can be fabricated by providing a pattern on a surface and promoting an electrostatic assembly of enzymes on surface in that pattern. Alternatively, the enzymes can be covalently attached to the surface in this pattern. In some cases, self-powered enzyme micropumps provided herein can have a fluid pumping speed that shows a substrate concentration- and reaction rate-dependent increase. In some cases, catalysis induced density-driven convective flow is the driving mechanism for the directional fluid pumping. In some cases, self-powered enzyme micropumps provided herein can be used to attain both spatial and temporal control over fluid transport, as well as delivery of colloids and small molecules. In some cases, self-powered enzyme micropumps provided herein can be triggered by the presence of specific analytes. In some cases, self-powered enzyme micropumps provided herein can be used with toxic analytes. For example, a toxic analyte can be drawn towards a self-powered enzyme micropump provided herein and be consumed as substrate, thereby reducing the ambient concentration of the toxic analyte (e.g., a phosphate-based nerve agent as a substrate for a phosphatase pump). In some cases, self-powered enzyme micropumps provided herein can include multi-enzyme cascades to provide regulation and microfluidic logic.

In some cases, self-powered enzyme micropumps provided herein can be used in a smart, micro- and/or nano-scale devices to control the direction and velocity of fluid and particle transport. In some cases, self-powered enzyme micropumps provided herein can remain viable and be capable of “turning on” even after prolonged storage.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DESCRIPTION OF DRAWINGS

FIG. 1A depicts a schematic showing enzyme pattern on a surface and triggered fluid pumping by enzymatic micropumps. (a) Au was patterned on a PEG-coated glass surface using an e-beam evaporator. The patterned surface was functionalized with a quaternary ammonium thiol, which forms a SAM (self-assembled monolayer) on the Au surface. The negatively charged groups on the enzyme bind selectively to the SAM-functionalized Au patterned surface via electrostatic assembly, resulting in an enzyme pattern on the surface. (b) Catalase enzyme immobilized on the Au pattern causes fluid pumping triggered by the presence of both glucose oxidase and glucose, which generates hydrogen peroxide in situ.

FIG. 1B depicts an enzyme-powered stimuli responsive autonomous release of cargo. General schematic showing functionalization of enzyme molecules on a positively charged (quaternary ammonium-terminated) hydrogel, followed by the triggered release of cargo (e.g. drug) in the presence of the enzyme substrate.

FIG. 1C depicts a glucose oxidase-powered stimuli responsive release of insulin. The solution concentration (units of μM) and percentage of insulin molecules released from a glucose oxidase-immobilized hydrogel as a function of time in the presence of different concentrations of glucose monitored using an UV-Vis spectrophotometer. The profile shows an increase in the amount of insulin released from the hydrogel with increasing glucose concentration in the surrounding solution. The observed behavior is a direct consequence of the enzymatic reaction-regulated fluid pumping.

FIG. 1D depicts an acid phosphatase-powered stimuli responsive release of 2-pralidoxime (2-PAM). The absorbance of the solution surrounding an acid phosphatase-immobilized hydrogel containing 2-PAM as a function of time in the presence of different concentrations of glycerophosphate (nerve agent simulant) monitored using an UV-Vis spectrophotometer. The profile shows an increase in the absorbance of the solution at 297 nm (absorbance wavelength at which 2-PAM absorbs in SAT buffer) with increasing glycerophosphate concentration in the surrounding solution. This increase in absorbance can be directly related with an increase in the concentration of 2-PAM released into the solution from the hydrogels. The observed behavior is a direct consequence of the enzymatic reaction-regulated fluid pumping.

FIG. 2 depicts a fluid pumping velocity in an enzyme-powered micropump as a function of substrate concentration and reaction rate. (a) Pumping velocity in a catalase-powered micropump increases in the presence of its substrate in a reaction rate-dependent fashion, at substrate concentrations ranging from 0.001 M to 0.1 M hydrogen peroxide. (b) Pumping velocity in a urease-powered micropump increases on increasing the substrate concentration from 0.001 M to 0.75 M urea. (c) Pumping velocity in lipase-powered micropump shows a concentration-dependent increase at substrate concentrations from 0.001 M to 0.5 M 4-nitrophenyl butyrate. (d) Pumping velocity in a glucose oxidase-powered micropump increases in a substrate concentration- and reaction rate-dependent manner from 0.001 M to 1 M glucose. The reaction rate calculations are based on k_{cat} (turnover number) and K_{M} (substrate concentration at which the reaction rate is half of the maximum rate for the system) values for enzymes in solution. Error bars represent standard deviations. The means and standard deviations are calculated for 30 tracer particles. The pumping velocities at different substrate concentrations are statistically different (P<0.01).

FIG. 3 depicts temporal and spatial changes in fluid pumping velocity for catalase-powered micropumps. The fluid pumping velocity in catalase-powered micropumps in presence of 50 mM hydrogen peroxide was monitored (a) 50-100 μm away from the enzyme pattern as a function of time at intervals of 30 minutes for a total duration of 4 hours, and (b) as a function of distance away from the Au pattern every 1000 μm for a total distance of 5000 μm. As shown, pumping velocity decreases over time and distance. Error bars represent standard deviations. The means and standard deviations are calculated for 30 tracer particles. The pumping velocities at different time intervals are statistically different from the pumping velocity at time t=5 mins (P<0.01). The pumping velocities at different distance intervals are statistically different from the pumping velocity at distance d=100 μm (P<0.01) (See Supplementary Information).

FIG. 4 depicts fluid pumping in enzyme micropumps generated by density-driven flows. (a) The fluid pumping velocity monitored in the upright and inverted pump setups showed no significant difference for any of the four enzyme micropumps. Error bars represent standard deviations. The means and standard deviations are calculated for 30 tracer particles. The pumping velocities monitored in upright and inverted pump setups are not statistically different (P>0.01). (b) The fluid pumping velocity monitored in the double-spacer (2× height of chamber, h) setup showed a ˜7-fold increase as compared to the single-spacer (h) setup for three enzyme micropumps. Error bars represent standard deviations. The means and standard deviations are calculated for 30 tracer particles. The pumping velocities monitored in single- and double-spacer setups are statistically different (P<0.01) (See Supplementary Information).

FIG. 5 depicts urease-powered stimuli responsive autonomous release of dye. (a) A general schematic showing functionalization of enzyme molecules on a positively charged (quaternary ammonium-terminated) hydrogel, followed by the triggered release of cargo in the presence of the enzyme substrate. (b) The concentration of dye (fluorescein) molecules (units of μM) released from urease-anchored hydrogel as a function of time in the presence of different concentrations of urea, monitored using an UV-Vis spectrophotometer. The profile shows an increase in the amount of dye released from the hydrogel with increasing urea concentration. The concentration of fluorescein dye molecules released was calculated from the absorbance values by using a calibration curve measured for the dye (Supplementary FIG. 16). The initial absorbance measurement was recorded at 30 min after substrate (urea) addition.

FIG. 6 depicts glucose oxidase-powered stimuli responsive release of insulin. The solution concentration (units of μM) and percentage of insulin molecules released from a glucose oxidase-immobilized hydrogel as a function of time in the presence of different concentrations of glucose monitored using an UV-Vis spectrophotometer. The profile shows an increase in the amount of insulin released from the hydrogel with increasing glucose concentration in the surrounding solution. The observed behavior is a direct consequence of the enzymatic reaction-regulated fluid pumping. The concentration of insulin released was calculated using molar extinction coefficient, ε₂₇₆=6,100 M⁻¹ cm⁻¹. The initial absorbance measurement was recorded at 10 min after substrate (glucose) addition.

FIG. 7 depicts TOC graphics of micropumps provided herein.

FIG. 8 depicts fluorescence imaging of SAM-modified Au surface in the presence and absence of fluorescent-labeled enzyme. (a) Fluorescence intensity was observed only on the Au pattern functionalized with SAM and dye-labeled enzyme, indicating that the enzyme binds selectively to the Au pattern and not on the PEG-coated glass surface. (b) No fluorescence intensity was observed when the SAM-modified Au pattern was not functionalized with enzyme.

FIG. 9 depicts temporal regulation of fluid pumping velocity in enzyme-powered

micropumps. The fluid pumping velocity monitored 50-100 μm away from the enzyme pattern as a function of time at intervals of 1 minute for a total time of ˜10 minutes showed no appreciable change in the velocity of the tracer particles for (a) a catalase-powered micropump in 50 mM hydrogen peroxide, (b) a urease-powered micropump in 0.75 M urea, (c) a lipase-powered micropump in 0.5 M 4-nitrophenyl butyrate, and (d) a glucose oxidase-powered micropump in 1 M glucose. Error bars represent standard deviations. The means and standard deviations are calculated for 30 tracer particles. The pumping velocities at different time intervals are not statistically different (P>0.01).

FIG. 10 depicts temporal velocity profile for catalase-powered micropump. The fluid pumping speed monitored 50-100 μm away from the enzyme pattern as a function of time at intervals of 1 minute for a total time of ˜10 minutes showed no appreciable change in the speed of the tracer particles for catalase-powered micropump in (a) 10 mM hydrogen peroxide and (b) 100 mM hydrogen peroxide. Error bars represent standard deviations. The means and standard deviations are calculated for 30 tracer particles. The pumping velocities at different time intervals are not statistically different (P>0.01).

FIG. 11 depicts temporal and spatial changes in fluid pumping velocity for urease-powered micropumps. The fluid pumping velocity in urease-powered micropumps in presence of 1 M urea was monitored (a) 50-100 μm away from the enzyme pattern as a function of time at intervals of 30-60 minutes for a total duration of ˜3.5 hours, and (b) as a function of distance away from the Au pattern every 1000 μm for a total distance of 5000 μm. Error bars represent standard deviations. The means and standard deviations are calculated for 30 tracer particles. The pumping velocities at different time intervals are statistically different from the pumping velocity at time t=5 mins (P<0.01). The pumping velocities at different distance intervals are statistically different from the pumping velocity at distance d=50 μm (P<0.01).

FIG. 12 depicts recharging of enzyme-powered micropumps. Pumping velocities

of (a) catalase- and (b) urease-powered micropumps were examined by introducing fresh substrate solution (50 mM hydrogen peroxide for catalase and 0.9 M urea for urease), after the initial substrate solution was exhausted and fluid pumping stops. The new pumping speeds were comparable to the ones observed in the first fluid pumping cycle. Error bars represent standard deviations. The means and standard deviations are calculated for 30 tracer particles. The pumping velocity after recharging is not statistically different from the pumping velocity at time t=5 mins (P<0.01).

FIG. 13 depicts spatial regulation of fluid pumping velocity in enzyme-powered micropumps monitored for shorter distances. The fluid pumping velocity monitored as a function of distance from the enzyme pattern showed no significant change in the velocity of the tracer particles for (a) a catalase-powered micropump in 50 mM hydrogen peroxide, (b) a

urease-powered micropump in 0.1 M urea, (c) a lipase-powered micropump in 0.5 M 4-nitrophenyl butyrate, and (d) a glucose oxidase-powered micropump in 1 M glucose. Error bars represent standard deviations. The means and standard deviations are calculated for 30 tracer particles. The pumping velocities at different distance intervals are not statistically different (P>0.01).

FIG. 14 depicts fluid pumping in enzyme micropumps monitored with positively and negatively charged tracers. The fluid pumping velocity monitored with positively charged amine-functionalized polystyrene tracers and negatively charged sulfate-modified polystyrene tracers showed no significant difference for all the four enzyme

micropumps. Error bars represent standard deviations. The means and standard deviations are
calculated for 30 tracer particles. The pumping velocities monitored with positive and negative tracers are not statistically different (P>0.01).

FIG. 15 depicts fluctuations in local fluid density in enzyme-powered micropumps. (a) Schematic representation of the design of an enzyme-powered micropump setup. (b) Sulfate-functionalized polystyrene tracer particles (2 μm) suspended in a 0.1 M PNB (4-nitrophenyl butyrate) or hydrogen peroxide solution (in 10 mM PBS) move towards the lipase- or catalase-functionalized Au pattern respectively (viewed below the pattern), thereby showing that the fluid is pumped upwards, when monitored in the vertical setup. (c) Sulfate-functionalized polystyrene tracer particles (2 μm) suspended in a 0.75 M urea solution (in 10 mM PBS) move towards the urease-functionalized Au pattern (viewed above the pattern). The tracers move away when viewed below the enzyme pattern, thereby showing that the fluid is pumped downward, when monitored in the vertical setup.

FIG. 16 depicts calibration curve of fluorescein dye. Plot showing the calibration curve of fluorescein dye measured using UV-Vis spectrophotometer. The calibration curve gives a direct correlation between absorbance and concentration of the dye.

FIG. 17 depicts urease-powered stimuli responsive autonomous release. The release of dye (fluorescein) molecules as a function of time from the urease-anchored hydrogel in the presence of different concentrations of urea, monitored using an UV-Vis spectrophotometer. The dye absorbance profile shows an increase in the amount of dye released from the hydrogel with increasing urea concentration, which is a direct consequence of enzymatic reaction-generated fluid pumping. The initial absorbance measurement was recorded at 30 min after substrate (urea) addition.

FIG. 18 depicts urease-powered stimuli responsive autonomous release. The release of dye (fluorescein) molecules as a function of time from urease-anchored hydrogels in the presence of different concentrations of urea, for two more trials (a) and (b), in addition to the one shown in the manuscript. The dye absorbance profiles for (a) urease anchored hydrogels in the presence of 0.005 M, 0.050 M and 0.500 M urea and (b) urease anchored hydrogels in the presence of PBS buffer, 0.05 M and 0.500 M urea show an increase in the amount of dye

released from the hydrogels with increasing urea concentration, which is a direct consequence of enzymatic reaction-generated fluid pumping. The initial absorbance measurement was recorded at 15 min after substrate (urea) addition.

FIG. 19 depicts glucose oxidase-powered stimuli responsive autonomous release of insulin. The release of insulin as a function of time from the glucose oxidase-immobilized hydrogel in the presence of different concentrations of glucose. The insulin absorbance profile shows an increase in the amount of insulin released from the hydrogel with increasing glucose concentration. The initial absorbance measurement was recorded at 10 min after substrate (glucose) addition.

FIG. 20 depicts glucose oxidase-powered stimuli responsive autonomous release of insulin. The release of insulin as a function of time from glucose oxidase-immobilized hydrogels in the presence of different concentrations of glucose, for two more trials (a) and (b), in addition to the one shown in the manuscript. The insulin absorbance profiles for (a) glucose oxidase anchored hydrogels in the presence of SAT buffer, 0.005 M, 0.050 M and 0.250 M glucose and (b) glucose oxidase anchored hydrogels in the presence of SAT buffer, 0.005 M, 0.050 M and 0.500 M glucose show an increase in the amount of insulin released from the hydrogel with increasing glucose concentration. The initial absorbance measurement was recorded at 10-15 min after substrate (glucose) addition.

FIG. 21 depicts contour plot of temperature distribution and streamlines of the convective flow. Contour plot showing (a) the distribution of the dimensionless temperature and (b) streamlines of the convective flow. Both the plots are reported for the experimental conditions (a=2.31).

FIG. 22 depicts variation of f(a) with a=R/h. Plot showing the variation of f(a) with a=R/h. Horizontal dashed line indicates the limit of large a.

FIG. 23 depicts density-driven fluid pumping in urease-powered micropump. (a) Schematic showing fluid flow in a device, analogous to a sink-reservoir model. The setup involves a small glass capillary tube filled with 1 M ammonium carbonate in buffer (product of urea hydrolysis), placed inside a larger capillary tube containing a buffered solution of 1 M urea. The system was completely closed such that flows could only generate through the opening of the small capillary inserted into the larger one. Sulfate-functionalized polystyrene microspheres (2 μm) were introduced as tracers to monitor the fluid flow. (b) On monitoring the tracer movement at the opening of the small capillary in the upright setup, fluid flow was outward at the capillary surface. The flow was inwards when viewed away from the surface in a different plane, by fluid continuity. (c) In the inverted setup, the direction of fluid flow reversed, that is, at the capillary surface, the tracers moved inwards. When viewed in a plane away from the surface, fluid flow was outwards.

FIG. 24 depicts schematic showing reaction steps involved in the synthesis of the SAM linker. Synthesis of quaternary ammonium thiol ligand, used for binding enzyme

molecules to the Au surface via electrostatic interactions.

FIG. 25 depicts synthesis of QDMAEMA-C4 monomer. (a) Schematic showing synthesis of QDMAEMA-C4 monomer. (b) 1HNMR spectra (D₂O) of the synthesized QDMAEMA-C4 monomer showing the expected proton resonances.

FIG. 26 depicts synthesis of polymeric hydrogel network. Schematic showing synthesis of polymeric hydrogel network from copolymerization of QDMAEMA-C4 monomer and N-isopropylacrylamide.

FIG. 27 depicts a characterization of polymeric hydrogel network by FT-IR. IR spectrum showed the characteristic signals from the different functional groups in the polymer hydrogel.

### DETAILED DESCRIPTION

A self-powered enzyme micropump provided herein can provide precise control over flow rate in response to specific signals. In some cases, self-powered enzymes micropumps provided herein can be ATP-independent. In some cases, self-powered enzyme micropumps provided herein can be non-mechanical. In some cases, self-powered enzyme micropumps provided herein can be surface-immobilized. In some cases, self-powered enzyme micropumps provided herein can include an enzyme selected from catalase, lipase, urease, glucose oxidase, and combinations thereof. In some cases, self-powered enzyme micropumps provided herein can provide a flow driven by a fluid density-gradient generated by an enzymatic reaction. In some cases, self-powered enzyme micropumps provided herein can increase the flow velocity with increasing substrate concentration and reaction rate. In some cases, self-powered enzyme micropumps provided herein can be triggered by the presence of specific analytes and can act as both a sensor and a pump. In some cases, self-powered enzyme micropumps provided herein can autonomously deliver small molecules and proteins in response to specific chemical stimuli. For example, self-powered enzyme micropumps provided herein can, in some cases, be used to release insulin in response to the presence of glucose.

In some cases, self-powered enzyme micropumps provided herein can include simple pattern of enzymes on a surface. In some cases, self-powered enzyme micropumps provided herein be fabricated by providing a pattern on a surface and promoting an electrostatic assembly of enzymes on surface in that pattern. In some cases, self-powered enzyme micropumps provided herein can have a fluid pumping speed that shows a substrate concentration- and reaction rate-dependent increase. In some cases, catalysis induced density-driven convective flow is the driving mechanism for the directional fluid

pumping. In some cases, self-powered enzyme micropumps provided herein can be used to attain both spatial and temporal control over fluid transport, as well as delivery of colloids and small molecules. In some cases, self-powered enzyme micropumps provided herein can be triggered by the presence of specific analytes. In some cases, self-powered enzyme micropumps provided herein can be used with toxic analytes. For example, a toxic analyte can be drawn towards a self-powered enzyme micropump provided herein and be consumed as substrate, thereby reducing the ambient concentration of the toxic analyte (e.g., a phosphate-based nerve agent as a substrate for a phosphatase pump). In some cases, self-powered enzyme micropumps provided herein can include multi-enzyme cascades to provide regulation and microfluidic logic.

In some cases, self-powered enzyme micropumps provided herein can be used in a smart, micro- and nano-scale devices to control the direction and velocity of fluid and particle transport. In some cases, self-powered enzyme micropumps provided herein can remain viable and be capable of “turning on” even after prolonged storage. In some cases, self-powered enzyme micropumps provided herein can be non-mechanical, self-powered nano/microscale pumps that precisely control flow rate and turn on in response to specific stimuli.

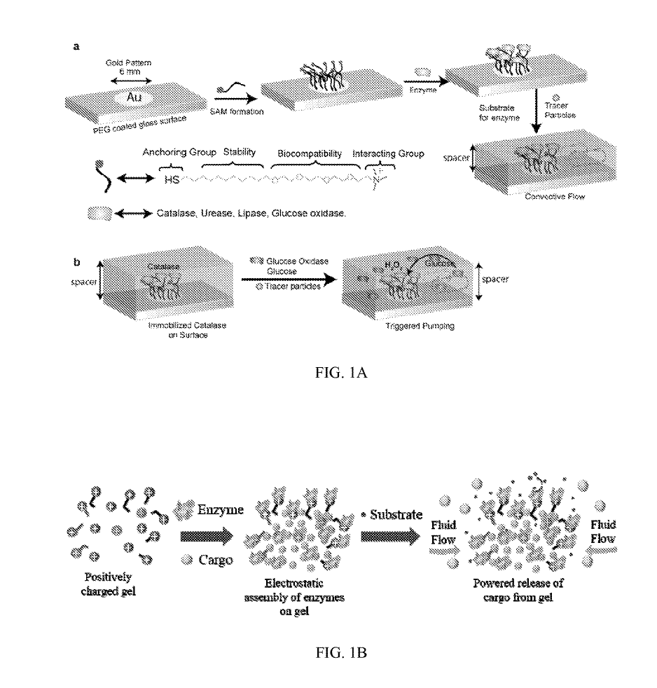

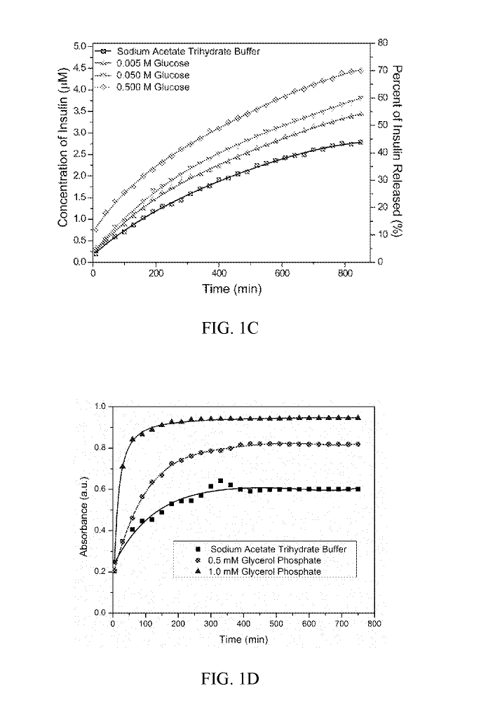

In some cases, self-powered enzyme micropumps provided herein can be cargo delivery devices, such as shown in FIG. 1B. For example, a cargo delivery device can be a drug delivery device. In some cases, a cargo delivery device can release insulin from a reservoir at a rate proportional to ambient glucose concentration (FIG. 1C). In some cases, a cargo delivery device can provide nerve agent decontamination and/or treatment. In some cases, a cargo delivery device provided herein can include an enzyme pump that uses nerve agents as fuel and releases an antidote in return (FIG. 1C). These self-powered pumps can remain viable and be capable of “turning on” even after prolonged storage.

In some cases, self-powered enzyme micropumps provided herein can be included in a sensor. For example, fluid speed depends on presence and concentration of analyte (e.g. biomarker, toxin) and/or factors like temperature, pH, and heat release. By using tracers or dyes to monitor fluid speed, a variety of analytes can be detected. This allows the design of inexpensive assays for the presence of specific analytes, or to measure the activity of an enzyme and its affinity for a specific analyte.

In some cases, self-powered enzyme micropumps provided herein can be used for bottom-up assembly and disassembly of dynamic structures. Since the enzyme pumps can pump particles suspended in a fluid, it is possible to form particle assemblies in specific locations by directional pumping. Furthermore, pumping can also be employed to disassemble such structures by directed transport of materials to specific places.

### Micropump Design and Enzyme Immobilization

Self-powered enzyme micropumps provided herein can be made using any suitable method. In some cases, a surface can be modified to create a pattern of an enzyme coating. For example, Au can be patterned on a PEG-coated glass surface using an e-beam evaporator. In some cases, an electron beam can be used to evaporate a thickness of 90 nm of Au on the PEG-functionalized surface, with a 10 nm adhesion layer of Cr. In some cases, the radius of the gold pattern can be 3 mm. In some cases, a surface can be cleaned prior to creating a pattern. For example, a PEG-coated glass surface can be cleaned with isopropanol followed by acetone and dried by blowing nitrogen.

After creating a pattern (e.g., of Au on PEG-coated glass), an enzyme can be used to form a self-assembled monolayer (SAM) on at least one surface. For example, previously synthesized quaternary ammonium thiol can form a self-assembled monolayer (SAM) on an Au surface. In some cases, the ligand can be dissolved in methanol and the surface can be incubated in it overnight at room temperature under an inert atmosphere, and optionally washed several times with methanol followed by PBS buffer, and dried under an inert atmosphere. In some cases, a SAM-modified surface can be incubated in an enzyme solution for multiple hours (e.g., 4-5 hours). In some cases, negatively charged enzymes can bind selectively to a thiol-functionalized Au patterned surface via electrostatic assembly. In some cases, an enzyme-functionalized surface can be washed with PBS to remove any unbound enzyme molecules from the surface. An enzyme-patterned surface can, in some cases, be covered with a secure-seal hybridization chamber (Electron Microscopy Sciences) with dimensions of 20 mm diameter and 1.3 mm height.

### Calculating Reaction Rate

The pumping velocities of the enzyme-micropumps provided herein were studied as a function of substrate concentration, which in turn, is related to the reaction rate of the catalytic reaction. The relation between substrate concentration and reaction rate is given by the Michaelis-Menten equation:

$$v = \frac{{V}_{max} [ S ]}{{K}_{M} + [ S ]} \text{:} {V}_{max} = {k}_{cat} [ E ] \qquad ( S1 )$$

where ν is the reaction rate, Vₘₐₓ is the maximum reaction rate achieved by the system and is defined as the turnover number (k_{cat}) multiplied for the enzyme concentration ([E]), [S] is the substrate concentration and K_{M} is the substrate concentration at which the reaction rate is Vₘₐₓ/2.

The reaction rate at each concentration of substrate was determined using the values of k_{cat} and K_{M} reported in the literature for each of the enzymes in solution; note that these values will be different for immobilized enzymes that are dimensionally restricted. It was assumed that the Au pattern was covered by a monolayer of quaternary ammonium linker-bound enzyme molecules in a tightly packed fashion. The enzyme concentration for each enzyme-powered micropump was determined by using the hydrodynamic radius of the enzyme, assuming that each enzyme in the pattern is spherical. The number of enzyme molecules on the Au pattern was determined from the surface area of the pattern (28.27 mm²) and cross sectional area of the respective enzymes.

Using Avogadro's number (6.02×10²³ molecules/mole), the moles of enzyme molecules were determined (5.7×10¹³ moles), and the concentration was then calculated using the volume of solution inside the spacer (4.084×10⁻⁷ m³).

### Illustration of Reaction Rate Calculation Using Catalase:

The radius of the gold patch is 3 mm. Therefore, its area is given by

π×(3×10⁻³)² m²=2.8×10⁻⁵ m² (=28.27 mm²)

Diameter of a single catalase molecule is 10.2 nm.

Cross sectional area of a single enzyme molecule:

$$\frac{π \times {( 10.2 \times {10}^{- 9} )}^{2}}{4} {m}^{2} = 8.2 \times {10}^{- 17} {m}^{2}$$

The approximate number of enzyme molecules in the gold pattern:

$$\frac{2.8 \times {10}^{- 5} {m}^{2}}{8.2 \times {10}^{- 17} {m}^{2}} = 3.4 \times {10}^{11}$$

$$Therefore , \\ [ E ] = \frac{5.7 \times {10}^{- 13} moles}{0.0004084 L} = 1.40 \times {10}^{- 9} M$$

Following Michaelis-Menten kinetics, for an enzyme concentration [E] and substrate

concentration, the reaction rate can be expressed as

$$v = \frac{4 {k}_{cat} [ E ] [ S ]}{{K}_{M} + [ S ]}$$

Here, we have considered four active sites per molecule of catalase. Now, for bovine liver catalase, K_{M}=93 mM. Therefore, at maximum substrate concentration, [S]=0.1 M=100 mM, the rate can be expressed as:

$$v = \frac{4 {k}_{cat} [ E ] [ S ]}{{K}_{M} + [ S ]} \\ {k}_{cat} ( catalase , per active site ) = 2.12 \times {10}^{5} {s}^{- 1} \\ v = \frac{4 ( 2.12 \times {10}^{5} {s}^{- 1} ) ( 1.40 \times {10}^{- 9} M ) ( 0.1 M )}{0.093 M + ( 0.1 M )} \\ v = 0.0006151 M . {s}^{- 1} \qquad [ 4 ]$$

The reaction rate can also be expressed as 2.51×10⁻⁷ moles·s⁻¹.

### Statistical Significance Analysis

To verify the statistical significance between two data sets, student's t-test was performed. The two-tailed P value was calculated using an unpaired t-test. The degree of freedom was 2n⁻² for all data sets, where n (30 tracer particles) is the number of independent measurements for each data set. The alpha level for all tests was chosen as 1% (0.01). When the result for a test of significance gave a P-value lower than 0.01 (alpha level), such results were referred to as statistically significant.

### Examples

Triggered fluid pumps using four different classes of enzymes were made. Gold (Au) was patterned on a polyethylene glycol (PEG)-coated glass surface. Next, the patterned surface was functionalized with a quaternary ammonium thiol, which formed a self-assembled monolayer (SAM) on the Au surface. On incubating the SAM-modified Au surface with enzyme, the negatively charged groups on the enzyme bind selectively to the modified Au surface via electrostatic self-assembly, resulting in an enzyme pattern on the glass surface (FIG. 1A, part a; Supplementary FIG. 8). To demonstrate the pumping ability of immobilized enzymes, a spacer (20 mm diameter; 1.3 mm height) was placed on top of the enzyme-patterned surface to seal the pump chamber and create a closed system. A buffered solution of substrate with suspended tracer particles was injected into the chamber and the fluid flow was monitored with an optical microscope.

### Pumping in Presence of Substrate

We examined catalase as our first example of an ATP-independent, enzyme-powered

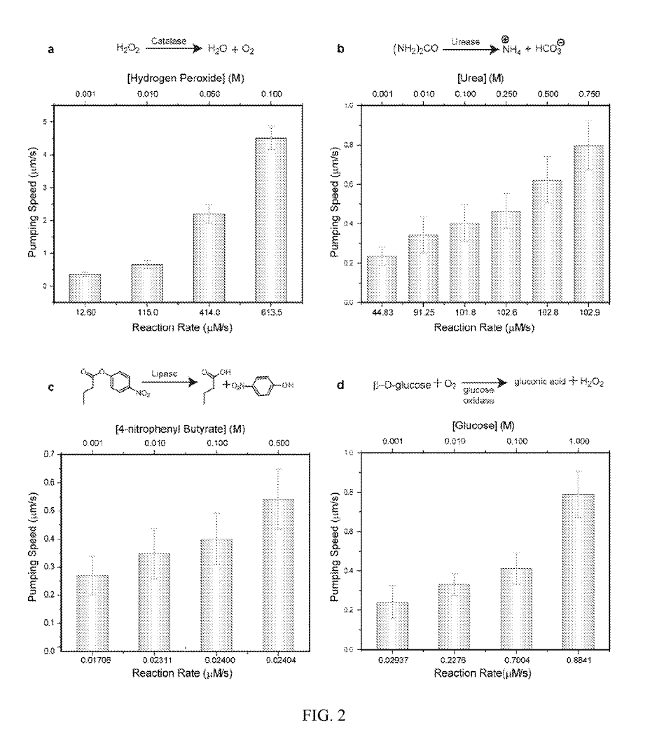

micropump. The enzyme was selectively immobilized on the Au pattern (6 mm diameter) as
described above and sulfate-functionalized polystyrene microspheres, 2 μm in size, were used as tracer particles to analyze the fluid flow. In the presence of substrate (hydrogen peroxide) the tracer particles moved towards the Au surface, indicating that the surrounding fluid is pumped inwards. Since the fluid flow was observed in a closed system, by
fluid continuity, fluid flow showed an outward motion when viewed above the enzyme-patterned surface. The fluid pumping velocity showed a substrate concentration and reaction rate dependent increase from 0.37 μm/s in 0.001 M hydrogen peroxide (enzymatic reaction rate (ν), 12.60 μM/s) to 4.51 μm/s in 0.1 M hydrogen peroxide (ν, 613.5 μM/s) (FIG. 2a). See above to see reaction rates are calculated. Note that the k_{cat} (turnover
number) and K_{M} (substrate concentration at which the reaction rate is half of the maximum rate for the system) values used are for enzymes in solution; these values will be different for
immobilized enzymes that are dimensionally restricted. No fluid pumping was observed in
absence of substrate. Similar pumping behavior was also observed for lipase and glucose oxidase (GOx) in the presence of their respective substrates, 4-nitrophenyl butyrate and glucose, with inward fluid flow near the Au surface and an outward flow when viewed above the surface. As with catalase, the pumping velocity increased with increasing substrate concentration and, in turn, enzymatic reaction rates in general (FIG. 2 c,d).

Opposite fluid flow was observed for urease anchored to the gold surface. Close to the glass surface, the tracer particles moved away from the Au pattern, indicating that the surrounding fluid was pumped outwards. When viewed up in the solution (away from the glass surface), by fluid continuity, an inward fluid flow was observed. As expected, the pumping velocity increased on increasing substrate concentration from 0.24 μm/s in 0.001 M (ν, 44.83 μM/s) urea to 0.80 μm/s in 0.75 M urea (ν, 102.9 μM/s) (FIG. 2b). No fluid pumping was observed in the absence of urea.

Enzyme-powered micropumps provided herein have the ability to sense substrate in the surrounding media and initiate fluid pumping in response. Using glucose and GOx, fluid pumping in the catalase pump was triggered by in situ generation of hydrogen peroxide (FIG. 1Ab). In the presence of 50 mM glucose and 0.1 μM GOx, with catalase immobilized on the Au pattern, the fluid was pumped inward at a speed of 1.2 μm/s. Pumping was not observed in absence of either glucose or GOx, or both. Thus, in principle, the enzyme pumps can be triggered by a variety of analyte molecules, opening up the possibility of designing enzyme-based devices that act both as sensor and pump.

### Temporal and Spatial Variations in Pumping

The temporal velocity profile was investigated for all four enzyme-powered pumps over both short and long time intervals. In case of catalase, fluid pumping was monitored for a time duration of 10 mins, at a distance of 50-100 μm away from the enzyme pattern and time intervals of 1 min. No significant change in velocity of tracer particles was observed at each of the three different concentrations of hydrogen peroxide −10 mM, 50 mM, and 100 mM within the 10 min time frame. Similar time-dependent studies of pumping speed

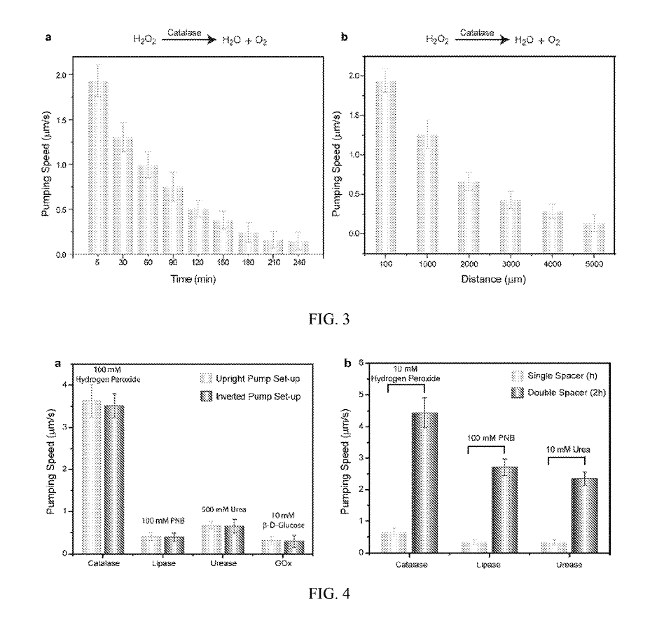

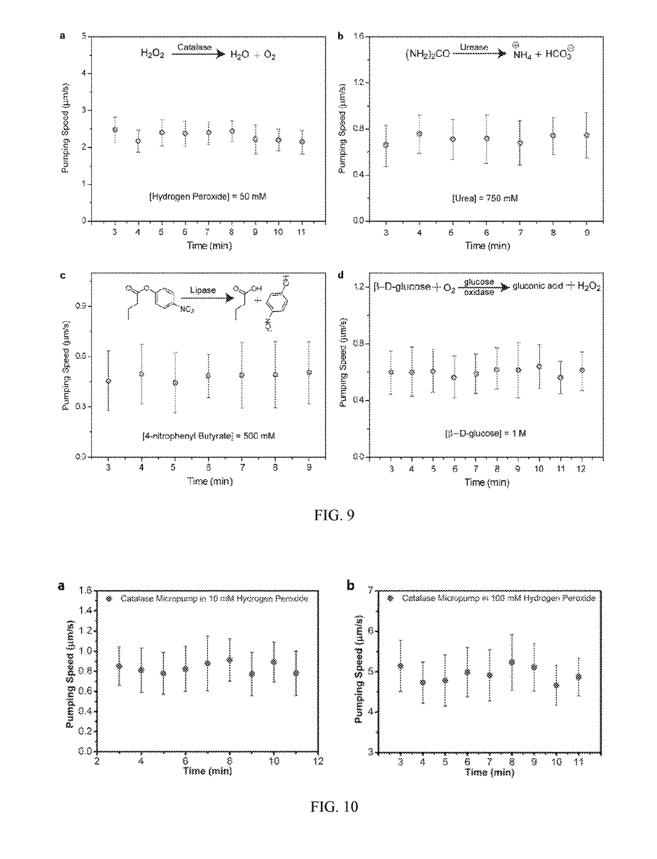

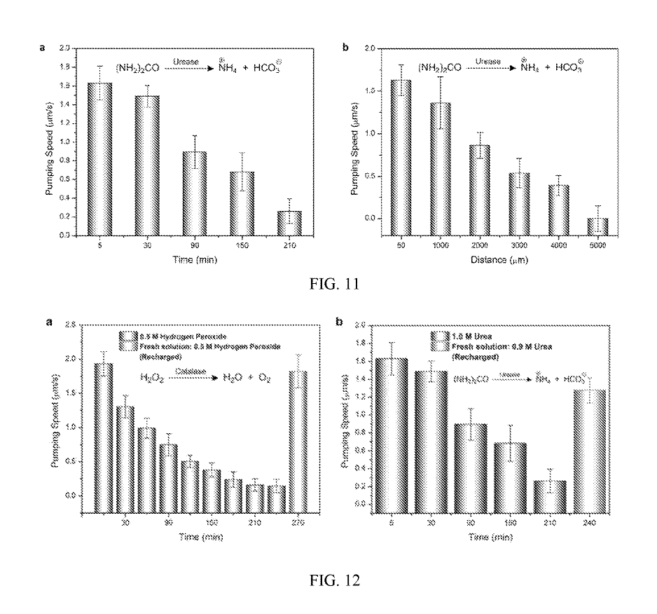

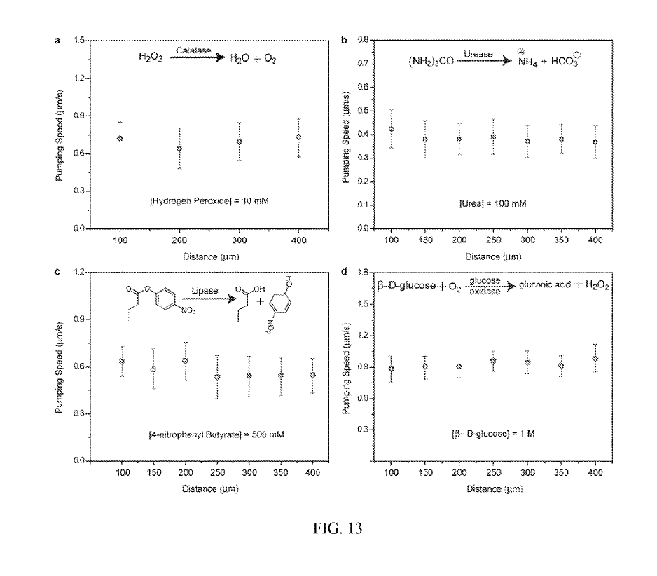

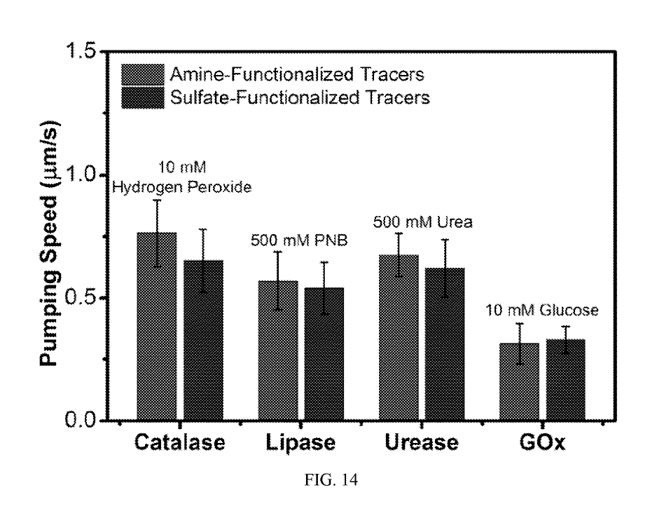

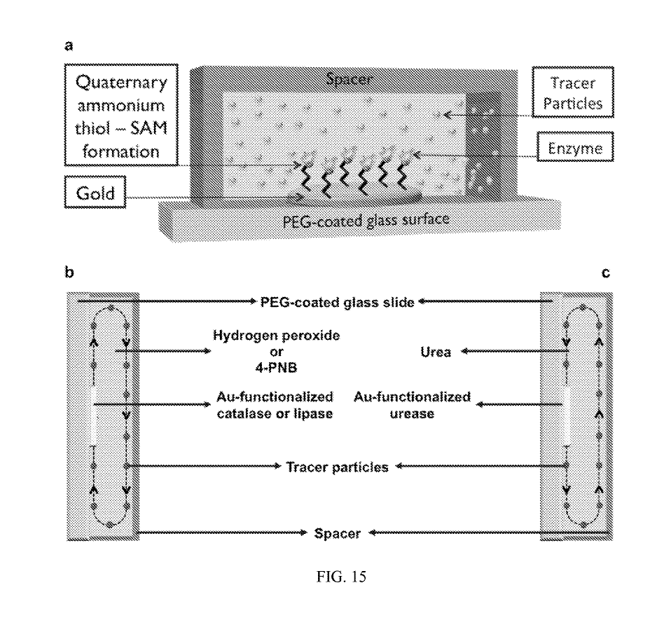

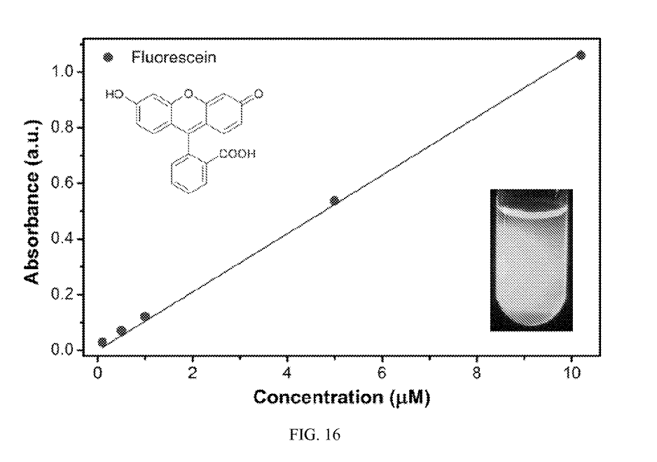

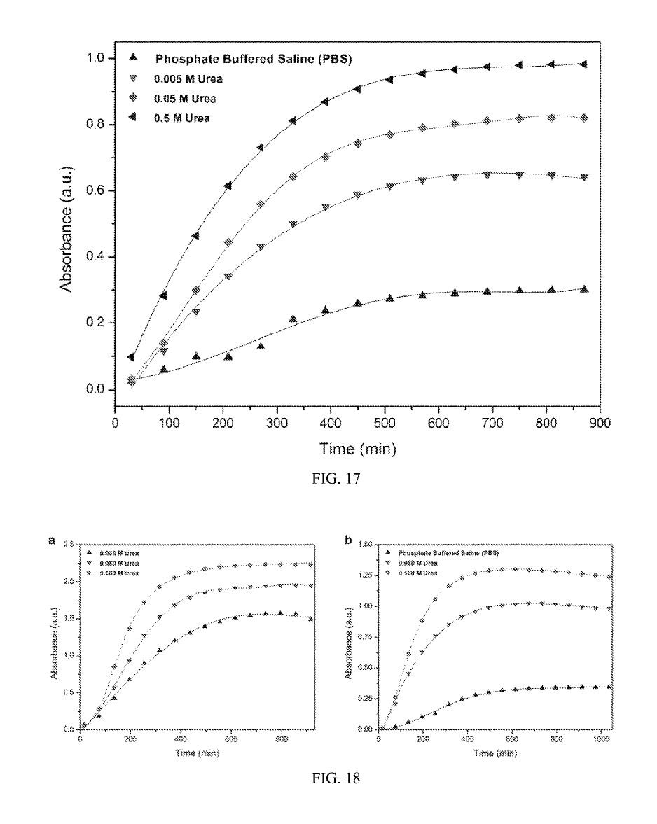

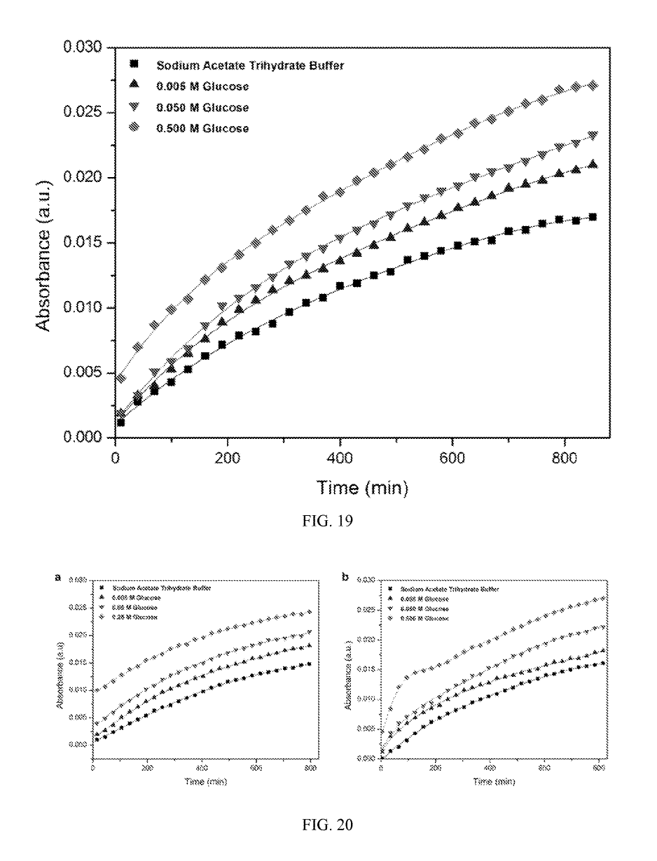

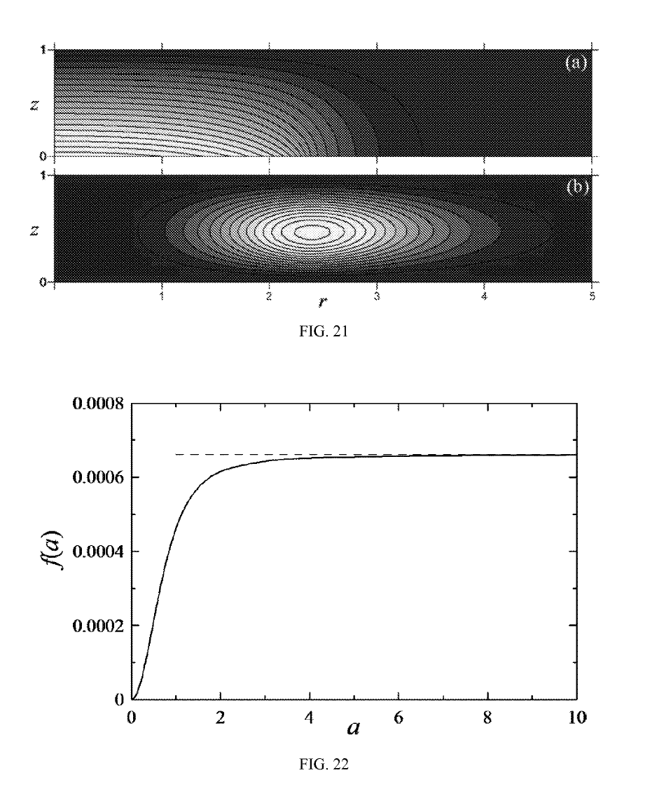

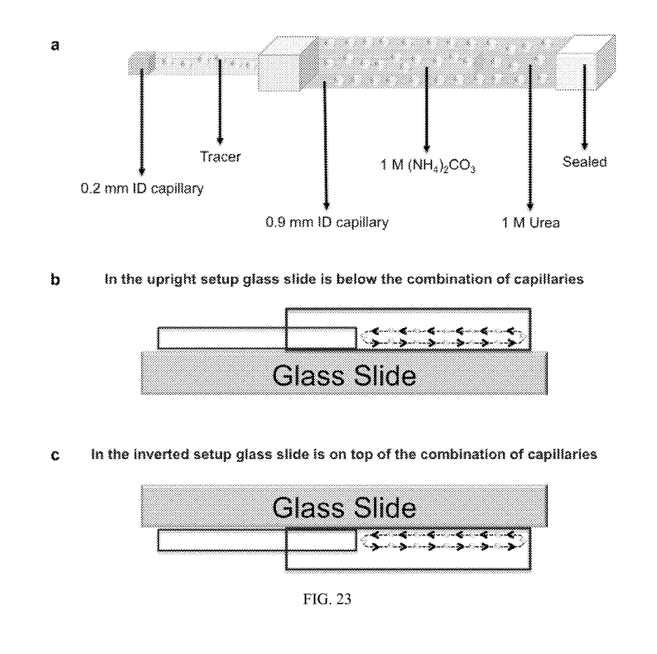

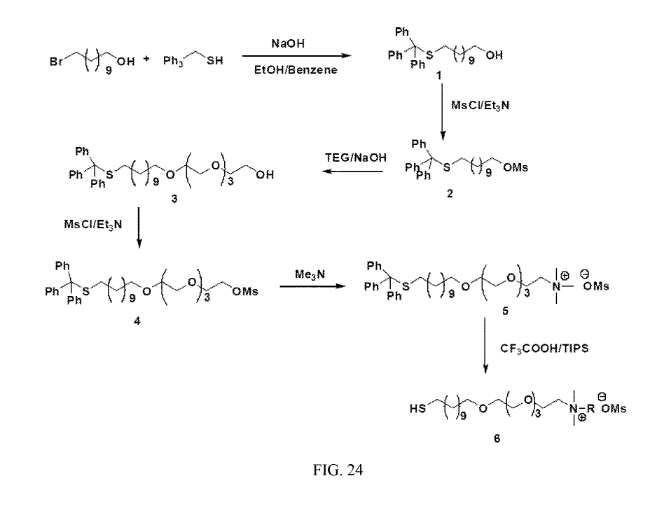

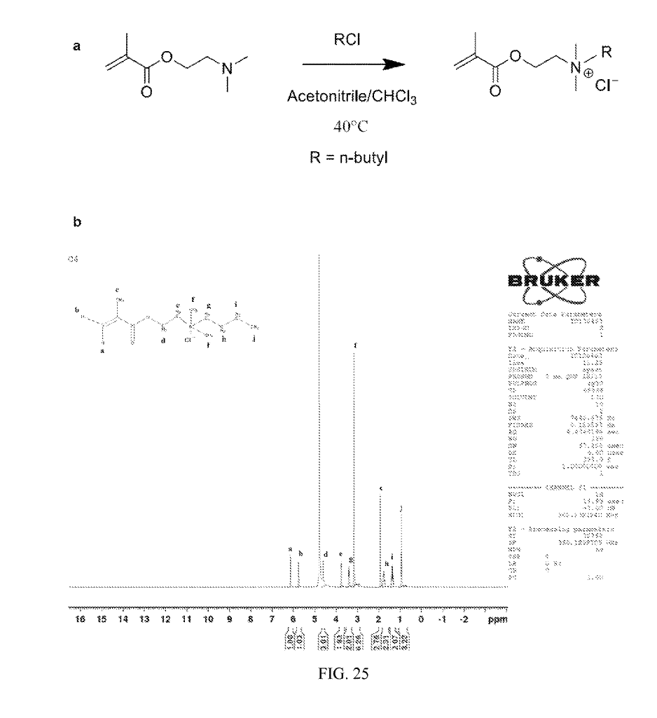

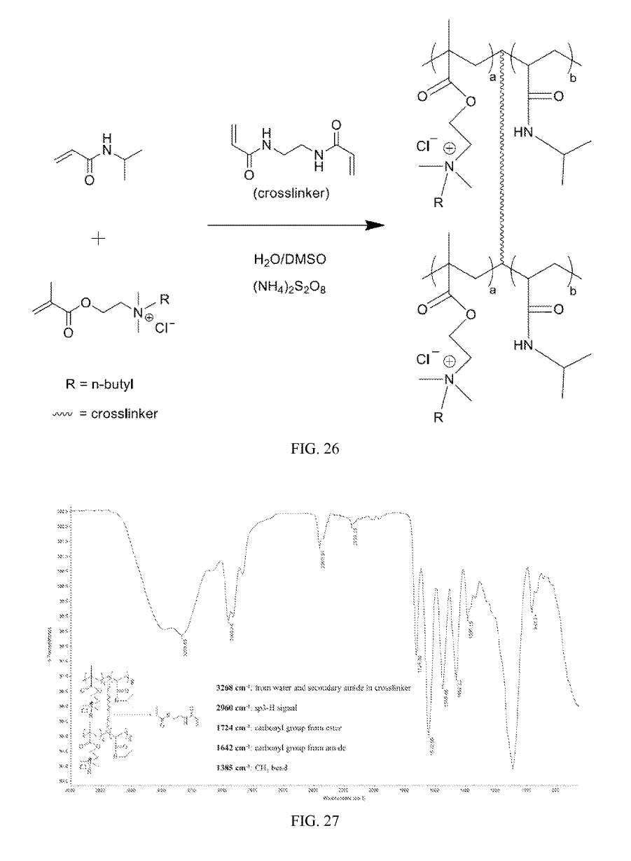

with urease in 0.75 M urea, GOx in 1 M glucose, and lipase in 0.5 M 4-nitrophenyl butyrate
showed no appreciable change in pumping velocity at short time intervals (FIG. 9). As expected, over longer time scales, the pumping velocity decreases. As the substrate is consumed, the reaction rate decreases, thereby slowing the fluid pumping speed. This was
demonstrated with catalase in presence of 0.050 M of hydrogen peroxide at regular time intervals of 30 mins, for a duration of 4 h (FIG. 3a). Similar behavior was observed with urease-powered pumps in the presence of 1 M urea (FIG. 11). Significantly, these pumps can be recharged by introducing fresh substrate solution, after the initial substrate solution is exhausted and fluid pumping stops (FIG. 12). For both catalase and urease, fluid pumping
resumed with a velocity similar to that observed previously at that specific substrate concentration.

The spatial velocity profile was also examined for each of these enzyme pumps. The fluid pumping velocity was examined at set distances moving away from the enzyme-functionalized Au pattern. At shorter distances (50-400 μm), the pumping velocities did not show significant variations for catalase-, urease-, GOx-, and lipase-powered pumps (FIG. 13).

As expected, the pumping velocity decreases at longer distances, as observed for catalase- (FIG. 3b) and urease-powered (FIG. 11) pumps monitored at distance intervals of 1000 μm, for an overall distance of 5000 μm.

### Pumping Mechanism

FIG. 2 suggests that pumping velocity is generally proportional to the reaction rate,

which in turn is controlled by both substrate concentration and inherent catalytic activity. A
detailed understanding of the mechanism will allow us to a priori predict the limits of reactive sensing and detection for specific analyte/pump combinations. It is possible to rule out several alternative mechanisms. Pumping arising from phoretic mechanisms such as
diffusiophoresis, osmophoresis, and self-electrophoresis has been demonstrated in the past
for surface-anchored catalytic particles. Symmetry breaking by anchoring of catalysts to solid surfaces can lead to chemical gradients due to the asymmetric production or depletion of solute molecules (charged or uncharged). Directional movement of tracers in the catalase-powered pump can arise from a non-electrolyte diffusiophoretic mechanism, due to a gradient caused by the conversion of hydrogen peroxide (two reactant molecules) to water and oxygen (three product molecules). However, such a mechanism can be ruled out from our observations with inverted pump set-up. When the experimental setup for the catalase-driven device was turned upside down such that the Au disk was on top, the direction of fluid flow relative to the glass surface was reversed. Fluid flowed outwards from the Au pattern at the glass surface, and by fluid continuity, moved in when viewed away from the surface. If indeed a non-electrolyte diffusiophoretic mechanism was in operation, the direction of fluid flow should remain the same irrespective of whether the pump device was upright or inverted.

Transport of fluid in urease-, lipase-, and GOx-powered pumps may be the result of

electrolyte diffusiophoretic mechanism, due to the generation of charged reaction products. Similar to its non-electrolyte counterpart, electrolyte diffusiophoresis can be ruled out from our observations with inverted pumps. In case of urease, the direction of fluid flow was reversed when the experimental set-up was turned upside down (Au disk on top). Closer to the surface the fluid flow was inwards, with tracers moving outwards when monitored away from the surface. Further, in case of both lipase and GOx, a similar effect was observed, i.e. the direction of fluid flow was reversed relative to the pump surface in the inverted setup. The zeta potential (surface charge) of the tracer particles has a profound effect on the direction of electrolyte diffusiophoretic transport; tracers with opposite charges move in opposite directions. The negatively charged sulfate-functionalized polystyrene tracers moved towards the enzyme-tethered gold pattern for lipase and glucose oxidase systems, and moved outwards for urease. If a diffusiophoretic mechanism was in operation, reversing the charge on tracer particles should reverse the direction of their movement. However, when positively charged amine-functionalized polystyrene tracers were used, the direction of their movement remained exactly the same as the negative tracers. Moreover, the speed of fluid pumping, monitored with positively charged tracers, was similar to their negative counterparts for all the enzyme pumps, thereby conclusively ruling out the possibility of a diffusiophoretic mechanism (FIG. 14).

As described above, the direction of fluid flow generated by all the four enzyme pumps reverse direction as the device cavity is inverted. The simplest explanation for this observation is a density-driven mechanism. The enzymatic reactions are exothermic and the temperature increase at the pump surface should give rise to thermal convection due to local decrease in fluid density. Thus, in an upright device the flow should be directed upward from the pump. Because of fluid continuity, near the glass surface the flow should be directed towards the Au pattern. For the inverted setup, the flow direction should be reversed because the lighter fluid tries to occupy the upper layers and spreads along the glass surface away from the Au pattern.

To validate our hypothesis, fluid flow was monitored in the inverted device to determine the pumping velocity. For all four enzyme-powered pumps, the pumping velocities in the inverted setup were similar to that in the upright one, strongly suggesting a density-driven mechanism as the governing factor (FIG. 4a). Further, the intensity of thermal convective flow within a horizontal layer of liquid in presence of a temperature gradient is governed by the Rayleigh number (Rₐ), such that,

$${R}_{a} = \frac{g β {h}^{4}}{v χ} \frac{d T}{d x} \qquad ( 1 )$$

Where g, h, β, v, and χ represent the gravitational acceleration, thickness of the liquid layer,
coefficient of thermal expansion, kinematic viscosity and heat diffusivity of the liquid, respectively. The magnitude of the vertical component of the temperature gradient

$$\frac{d T}{d x}$$

can be estimated by calculating the heat flux (in Jcm⁻²s⁻¹) as

$$\frac{d T}{d x} ≃ \frac{Q}{κ} ,$$

where, κ is the thermal conductivity of the liquid. The heat flux depends on the rate r and enthalpy ΔH of the chemical

reaction as follows:

$$Q = \frac{R Δ H}{π {R}^{2}} \qquad ( 2 )$$

Assuming the flow to be steady and small in magnitude, the speed can be scaled as:

$$V \text{:} \frac{χ}{h} {R}_{a} f ( a ) \qquad ( 3 )$$

where, the function f(a) depends on the aspect ratio of the micropump, a=R/h, where R is the radius of the pump surface. The flow, therefore, can be characterized by a speed given by:

$$V ∼ \frac{g β {h}^{3} r Δ H}{v κπ {R}^{2}} f ( a ) \qquad ( 4 )$$

At small Rₐ the function f(a) can be found solving two uncoupled boundary-value problems: first to derive the temperature of the fluid solving the Laplace equation with the prescribed heat flux at the reactive patch and constant temperature at the upper plate. Then the fluid velocity can be found via the linearized Navier-Stokes-Boussinesq equation. To reiterate, within this linear model, f(a) only changes its sign, when the gravity is inverted.

Numerical calculations within this approach showed that f(a) grows from zero up to a value of 10−3, saturating beyond R>3 h. Therefore, for R>3 h, any increase in the layer thickness h should result in an increase in the flow speed proportional to h3. For smaller a, the prefactor f(a) slightly diminishes this effect. For example, for the experimental setup with a=2.3 (R=3 mm, h=1.3 mm), the velocity grows by a factor of 6.6, when the layer thickness is doubled. For three different enzymes, the speed increased approximately by a factor of 6.8, confirming our theoretical proposition (FIG. 4b). Further, assuming the values of reaction rate (r=10⁻⁷ moles s⁻¹), enthalpy (ΔH=100 kJ mole−1) and height of the cavity (h=1 mm) to be of the same order of magnitude for all the enzymes, the speed was determined as ˜1 μm/s, in good agreement with the experimental results. Interestingly, even though the Rayleigh number for our system is fairly high (˜10), the smaller magnitude of f(a) helps in keeping the flow speed linear in Ra.

In case of urease, the observed effect is opposite from the expected one. Although the enzyme catalytic reaction is exothermic, the fluid is pumped outwards in the upright device. We hypothesized that since the products of urea hydrolysis are all ionic (NH₄⁺ and HCO₃⁻), these solvated ions can increase the density of the fluid near the enzyme pattern. This local increase in density causes the fluid to spread along the glass surface resulting in a density-driven convective flow, directing the fluid away from the pattern. In the inverted setup, the denser fluid generated on the top of the device settles down to lower layers in the cavity, and by fluid continuity drives the fluid flow inwards near the glass surface. Therefore, in this case the fluid density can be written as:

ρ=ρ₀(1−β(T−T₀)+β_{C}(C−C₀)) (5)

where, ρ is the final fluid density, T is the absolute temperature, C is the concentration of reaction products, ρ₀, T₀, and C₀ are the reference values of these three characteristics, is the volumetric temperature expansion coefficient, is the solute's coefficient of expansion. For the fluid density grows as the concentration of products increases. Therefore, for urease the situation is more complicated and double diffusive convection sets in with the competing impacts of the reaction on the flow density and hence, on the flow. In order to verify our hypothesis, the fluid flow was examined in two different systems.

The movement of tracer particles was monitored for the urease pump in a vertical device setup (FIG. 15). The fluid flowed downwards when viewed both below and above the enzyme-patterned surface, indicating an overall downward flow at the enzyme-patterned surface in the vertical setup. Again, by fluid continuity, the fluid flowed upwards away from the surface. The reaction-generated products being denser than the reactants settle to the bottom layers of the device, thereby driving the fluid flow downwards. The mechanism proposed for urease-powered micropump can also be established by monitoring the fluid flow using a sink-reservoir model (see Supplementary Information). Similar experiments using vertical setups were performed with catalase- and lipase-powered micropumps (FIG. 15). In both of these cases, the fluid flow was upwards when viewed below the Au pattern close to the surface, against gravity. This upward fluid movement also supports the mechanism proposed previously: convective flows resulting from a thermal gradient. The increase in temperature at the pump surface due to these enzyme catalytic reactions decreases the local fluid density for catalase and lipase, thereby driving the fluid flows upwards. The effect of temperature on solute-particle interaction may also play a role in the observed pumping.

### Substrate-Triggered Release of Molecules

The ability of enzyme-powered micropumps to respond to an external stimulus (e.g.

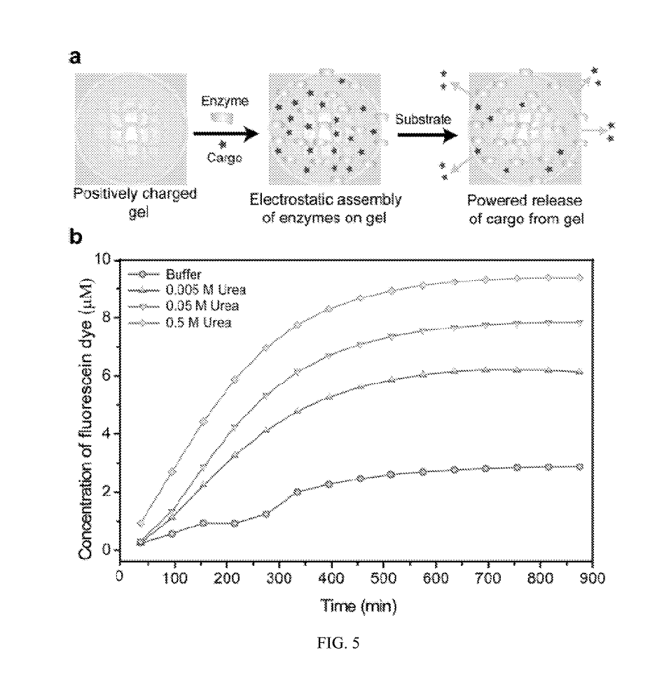

substrate) and produce a change in the surrounding environment by generating fluid flows makes them suitable candidates for applications like drug delivery, where a controlled response to an external stimulus is required to attain a specific goal, such as triggered administration of a drug. We fabricated a proof-of-concept design to demonstrate the potential ability of the enzyme pumps as autonomous stimuli-responsive drug delivery devices. Positively charged hydrogels were used as scaffolds for immobilizing enzymes, as well as reservoirs for small molecules. We anticipated the active release of small molecules and proteins from the hydrogel in presence of the enzyme substrate via a self-pumping mechanism. Hydrogels with quaternary ammonium functionality were synthesized and used as the template for enzyme immobilization via electrostatic self-assembly, similar to the previous pump setup. Since the hydrogel also serves as a reservoir for small molecules (cargo), simultaneous incubation of the hydrogels with enzyme and cargo molecules (to be released), led to their absorption in the gel network. The release of fluorescein dye molecules (used as a model cargo) as a function of time was monitored from urease-immobilized hydrogel in presence of varying urea concentrations (FIG. 5) using a UV-Vis is spectrophotometer. While there was some leaching of dye molecules through passive diffusion in the absence of any substrate, the dye release rate from the hydrogel increased with increasing substrate concentration (FIG. 5b, FIGS. 17-18). This is a direct consequence of enzymatic reaction regulated fluid pumping. To maintain a stable solution pH, all measurements were performed in phosphate buffered saline (PBS buffer). This ensured that the enzymatic activity was retained and that absorbance analyses were not subject to changes in solution pH, since fluorescein is known to show a pH-dependent change in absorbance.

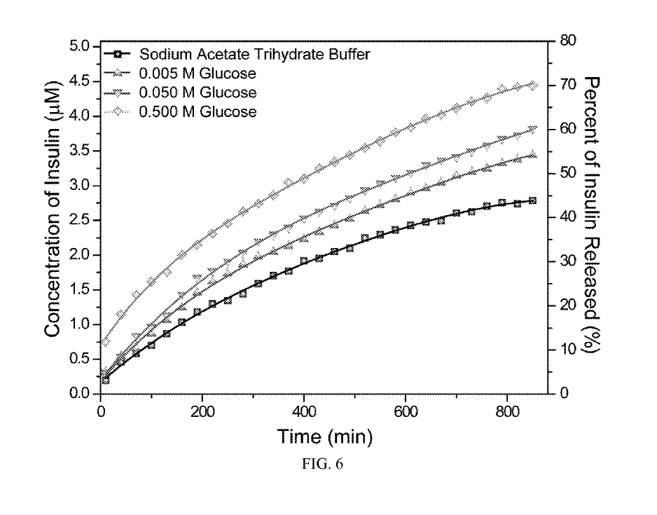

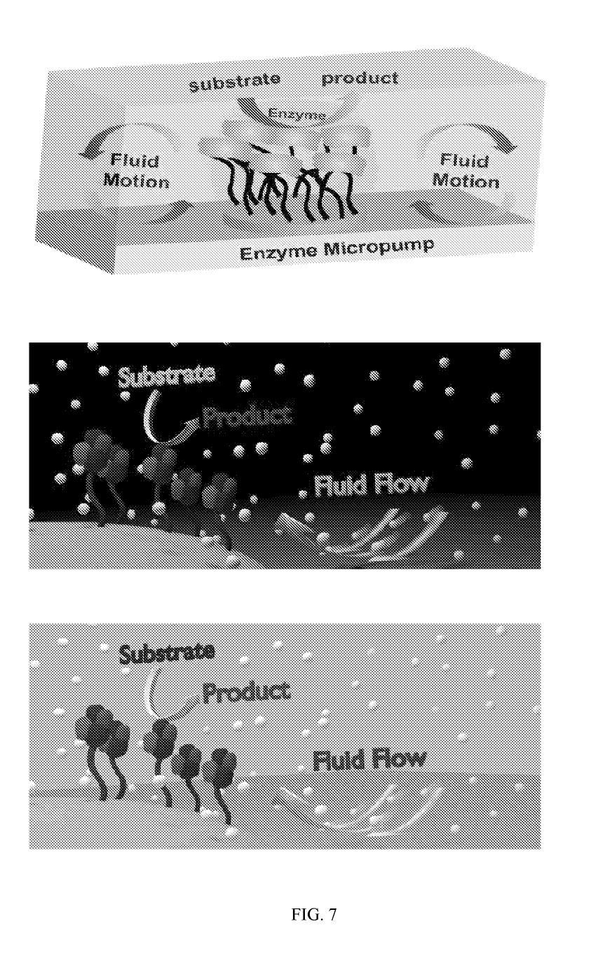

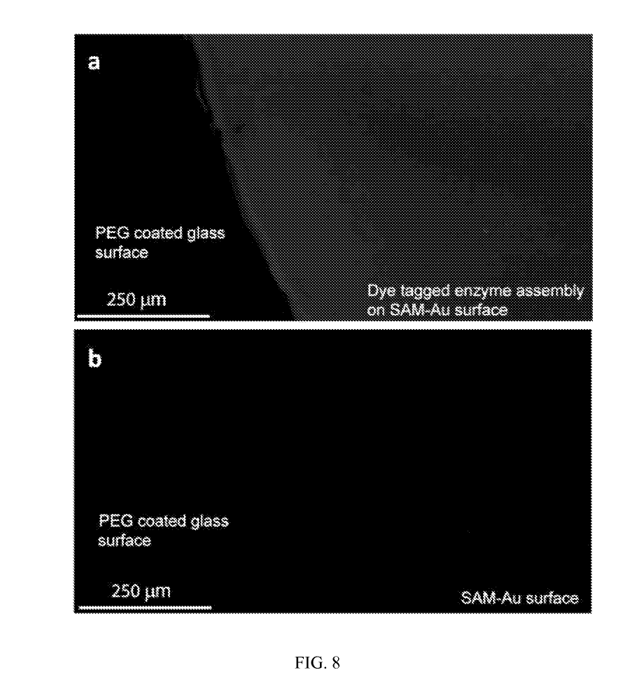

In another proof-of-concept demonstration, the release of insulin from glucose oxidase immobilized hydrogels was shown at different concentrations of glucose in sodium acetate trihydrate (SAT) buffer (pH 5.23). Increasing levels of insulin release from the hydrogel were achieved with increase in glucose concentration in the surrounding solution (FIGS. 6 and 19-20). The release profile for insulin is somewhat different from that of the dye, presumably because of differences in interaction with the host hydrogel. Our results suggest the design of a rechargeable enzyme pump that can actively release insulin at a rate proportional to ambient glucose concentration. Note that one of the glucose concentrations employed (0.005 M) is in the physiologically relevant range. The autonomous delivery device described above contrasts with other recently described systems, which involves the passive release of insulin due to scaffold decomposition. Currently, we are exploring this approach with different enzymes and hydrogel systems. We assume that the structural variation of hydrogel is a key parameter for sustained release of molecules from the cross-linked gel network.

### Other Embodiments

It is to be understood that, while the invention has been described herein in conjunction with a number of different aspects, the foregoing description of the various aspects is intended to illustrate and not limit the scope of the invention, which is defined by the scope of the appended claims. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

1. A drug delivery device adapted to release a drug in response to an analyte comprising:
a. a reservoir comprising said drug; and
b. an enzyme positioned adjacent to said reservoir, the enzyme being adapted to catalyze a reaction of said analyte to drive a fluid flow adjacent to said reservoir to increase a release of said drug from said reservoir.

2. The drug delivery device of claim 1, wherein said reservoir is a hydrogel.

3. The drug delivery device of claim 2, wherein said hydrogel is a positively charged hydrogel.

4. The drug delivery device of claim 2, wherein said hydrogel is a quaternary ammonium-terminated hydrogel.

5. The drug delivery device of claim 2, wherein said enzyme is bound to a surface of said hydrogel.

6. The drug delivery device of claim 1, wherein said drug is insulin and said analyte is glucose.

7. The drug delivery device of claim 6, wherein said enzyme is glucose oxidase.

8. The drug delivery device of claim 1, wherein the analyte is a nerve agent and the drug is an antidote for said nerve agent.

9. The drug delivery device of claim 1, wherein said enzyme catalyzes a decomposition reaction of said analyte.

10. A sensor for an analyte comprising:
a. an enzyme bound to a surface, the enzyme adapted to catalyze a reaction of said analyte to drive a fluid flow adjacent to said surface; and
b. a flow meter to detect a flow of fluids adjacent to said surface.

11. The sensor of claim 10, further comprising a processor to calculate a concentration of said analyte from the detected flow of fluids adjacent to said surface.

12. The sensor of claim 10, wherein said enzymes are patterned on said surface.

13. The sensor of claim 10, wherein said enzyme is bound to a self-assembled monolayer on the surface.

14. The sensor of claim 10, wherein said surface comprises a PEG-coated glass surface having a pattern of gold.

15. The sensor of claim 10, wherein the enzyme is selected from the group consisting of catalase, lipase, urease, glucose oxidase, and combinations thereof.

16. A self-powered enzyme micropump comprising a pattern of one or more enzymes on a surface, the pattern of enzymes being adapted to provide precise control over at least one fluid flow rate in response to a specific analyte.

17. The micropump of claim 16, wherein the self-powered enzyme micropump is ATP-independent.

18. The micropump of claim 16, wherein the self-powered enzyme micropump is non-mechanical.

19. The micropump of claim 16, wherein the self-powered enzyme micropump comprises catalase, lipase, urease, glucose oxidase, or a combination thereof.

20. The micropump of claim 16, wherein the self-powered enzyme micropump provides a flow driven by a fluid density-gradient generated by an enzymatic reaction.

21. The micropump of claim 16, wherein the self-powered enzyme micropump increases a flow velocity with increasing substrate concentration and reaction rate.

22. The micropump of claim 16, wherein the self-powered enzyme micropump is adapted to be triggered by the presence of a specific analyte.

23. The micropump of claim 16, wherein the self-powered enzyme micropump acts as a sensor.

24. The micropump of claim 16, wherein the self-powered enzyme micropump is adapted to autonomously deliver small molecules, proteins, or a combination thereof in response to specific chemical stimuli.

25. The micropump of claim 24, wherein the self-powered enzyme micropump is adapted to release insulin in response to the presence of glucose.

26. The micropump of claim 24, wherein the self-powered enzyme micropump is a phosphatase pump adapted to be triggered by a phosphate-based nerve agent.

27. The micropump of claim 16, wherein the pattern of enzymes is assembled on the surface.

28. The micropump of claim 16, wherein the self-powered enzyme micropump is adapted to provide a multi-enzyme cascade.

29. The micropump of claim 16, wherein the catalyst is formed on a PEG-coated glass surface on a pattern of Au.
